(19) United States
(12) Patent Application Publication
(10) Pub. No.: US 2025/0367351 A1
(43) Pub. Date: Dec. 4, 2025

(54) Breast Pump

(71) Applicant: MEDELA HOLDING AG, Baar (CH)

(72) Inventors: Jakub Pawlowski, Zug (CH); Danielle Prime, Bateau Bay (AU)

(21) Appl. No.: 18/874,523

(22) PCT Filed: Jul. 3, 2023
(86) PCT No.: PCT/EP2023/068249

§ 371 Date: Dec. 12, 2024

(30) Foreign Application Priority Data
Jul. 4, 2022 (EP) 22182874.2

(51) Int. Cl. A61M 1/06

(52) U.S. Cl. CPC A61M 1/06; A61M 2205/586

(57) ABSTRACT

A breast pump shaped at least in part to fit inside a bra with an outer shell and a breast shield element including a nipple tunnel adapted to receive a nipple of the lactating mother, which breast pump provides sufficient space within the outer shell for arranging the functional components of the breast pump within the outer shell while meeting the anatomical circumstances posed by the user of the breast pump.

The outer shell of the breast pump projects a frontal orthographic plane that is shaped like an oval, which oval is defined by a convex boundary encircling three circles, a first top circle, a first bottom circle and a second bottom circle, wherein a radius (RB) of the of the first bottom circle and a radius (RB) of the second bottom circle are equal and the ratio of a radius (RT) of the first top circle to the radius (RB) of the first or the second bottom circles is between 0.3 and 0.8.

### CROSS-REFERENCE TO RELATED APPLICATION

The present application is the US national phase of International Patent Application No. PCT/EP2023/068249, filed Jul. 3, 2023, which claims priority to European Application No. 22182874.2, filed Jul. 4, 2022. The priority application, EP 22182874.2, is hereby incorporated by reference in its entirety.

### TECHNICAL FIELD

The present invention relates to a breast pump shaped at least in part to fit inside a bra and comprising an outer shell, which outer shell at least in part receives a breast shield, specifically a nipple tunnel of said breast shield, which nipple tunnel is adapted to receive a nipple of the breast. Such breast pump is used for expressing milk from lactating mothers.

### BACKGROUND

So-called in-bra pumps, which at least in part fit into the bra and thus can be operated without necessarily holding the breast pump are well adapted in the marked.

WO 2018/229508 A1 (Chiaro) discloses such breast pump with a housing defining an outer shell and a closure cap attached to the outer shell, which closure cap has an opening for introducing the nipple tunnel into the housing. The outer shell of this prior art document is in part defined by a milk container, which is arranged below the housing. When being assembled, the breast shield essentially is enclosed by the housing. A flange portion of the breast shield rests against the housing, which for this to attain has a concave supporting surface for the breast shield. The housing of this prior art document also receives a battery for energizing a pump, which pump is arranged within the housing. Thus, the prior art breast pump contains all functional components for operating the breast pump and expressing milk. Control can e.g. be attained by a handheld device connected to the breast pump via Bluetooth.

WO 02/102437 A2 (Whisper Wear) discloses a breast pump shaped in part to fit inside the bra comprising an outer shell surrounding the nipple tunnel and at least one valve as well as a motor and a battery for energizing the motor. Expressed milk is released through a tube into a bag which is suspended below the outer shell and is not surrounded by the bra.

EP 2 280 745 A1 (Garbez) discloses a further example for a breast pump shaped at least to fit inside the bra. In this prior art document, the pump which provides the negative pressure to express milk from the breast is suspended by a belt of the user. Thus, the pump is not received within the bra. However, the nipple tunnel and means for directing flow from the breast into a milk container, which milk container is at least in part defined by the outer shell, are received within the bra. A respective disclosure is contained in U.S. Pat. No. 8,702,646 (Garbez).

WO 2019/226504 A1 (Exploramed) discloses a pump comprising an outer shell, which houses a battery and a motor for generating negative pressure within the housing of the pump. The housing likewise comprises a plastic bag which receives the milk expressed.

US 2013/0023821 A1 (Khalil) discloses a breast pump with an outer shell which surrounds the nipple tunnel and a valve for directing the expressed milk. In one embodiment, a tube is connected to the breast pump which leads the expressed milk to a container outside of the bra. In another embodiment, a milk container is attached to a housing which contains the pump and a battery for energizing the pump and which container at least in part is defined by the outer shell of the breast pump.

All the above discussed pumps are to be fitted into the bra. The dimensions of the outer shell are selected such that appropriate space can be provided for those components being supported by the bra, while leaving sufficient volume e.g. for receiving the expressed milk in case the amount of expressed milk is likewise supported by the bra, i.e. the milk container is surrounded by the outer shell forming at least in part the housing. On the other hand, the space within the bra is limited. While some portions of the breast pump supported by the bra may project beyond the textile material defining the bra, the above designs aim to shape the breast pump to as best possibly imitate the anatomic shape of the bust.

However, the anatomic reality of the female chest provides limited space for arranging the breast pump in contact with one breast. Usually, two individual breast pumps are provided for the left and the right breast, which may be energized or operated by an external common pump. Nevertheless, as two breast pumps are worn simultaneously, their spatial extension is limited to one half of the female breast.

The present invention aims to provide a breast pump shaped at least in part to fit inside a bra with an outer shell and a breast shield element including a nipple tunnel adapted to receive a nipple of the lactating mother, which breast pump provides sufficient space within the outer shell for arranging the functional components of the breast pump within the outer shell while meeting the anatomical circumstances posed by the user of the breast pump. In particular, the present invention wishes to provide a proposal for a specific shape of the outer shell to comply with the above demands.

### SUMMARY OF THE DISCLOSURE

As a solution to the above problem, the breast pump of the present invention has an outer shell which projects a frontal orthographic plane with a specific oval shape. Respective oval shape of the outer shell in the frontal orthographic plane is defined by a convex boundary to encircling three circles. The breast pump is comfortably wearable inside a bra.

Those three circles include a first top circle, a first bottom circle, and a second bottom circle. The radius of the first bottom circle and the radius of the second bottom circle are equal and the ratio of a radius R_{T} of the first top circle and each of the radii R_{B} of the first and the second bottom circles is between 0.3 and 0.8.

Respective circles are to define the shape of the shell in the frontal orthographic plane for the top and the bottom of the outer shell in the frontal orthographic plane. The overall shape of the outer shell in the frontal orthographic plane preferably is not concave i.e. exclusively convex or straight and convex. As the first and the second bottom circle are larger than the first top circle, the outer shell projects the frontal orthographic plane in a form essentially corresponding to an avocado.

The first and the second bottom circle may have the same center. In other words, the bottom of the outer shell may project the frontal orthographic plane in a form which is defined by a single circle. Alternatively, a center of the first bottom circle and a center of the second bottom circle are positioned on a lateral symmetry line X at a distance equal to a value between 0% and 15% of the radius of the first or the second bottom circle. As mentioned previously, the first and the second bottom circle are usually identical. Due to this offset in the two centers for the first and the second bottom circle, the bottom section of the outer shell provides more space for arranging components of the breast pump, such as the motor, a battery, or a control of the motor, or may provide e.g. an enlarged volume for defining a milk container or milk chamber at least partially defined by the outer shell. As in prior art previously discussed, such milk container may be provided by or near the bottom section of the outer shell. Such milk container may as well be completely surrounded by the outer shell with the nipple tunnel projecting into the milk container, thereby reducing the overall volume for storing expressed milk within the milk container.

On a regular basis, the longitudinal symmetry line defines two identical halves of the outer shell projecting the frontal orthographic plane. Naturally, the longitudinal symmetry line lies within this frontal orthographic plane or extends at least parallel thereto. The description of the invention at least in part refers to the lateral symmetry line. This lateral symmetry line extends in the same plane as the longitudinal symmetry line with the longitudinal symmetry line and the lateral symmetry line being perpendicular to each other. Those two lines intersect as a point which may preferably be associated with both centers of the first and the second bottom circles. Respective symmetry line may likewise intersect at a middle point between the centers of the first and the second bottom circles, with the middle point being disposed equidistant from the respective centers of the first and the second bottom circles.

Both alternatives provide a shape of the outer shell projecting the frontal orthographic plane, which shape is symmetrical to the longitudinal symmetry line.

Preferably, the center of the first top circle is positioned on the longitudinal symmetry line at a distance greater than the difference between the radius of the first or the second bottom circle and the radius of the first top circle and less than the sum of the radius of the first top circle plus the radius of one of the first and the second bottom circles.

Preferably, the convex boundary is not concave, i.e. exclusively convex or convex and straight. In other words, the outer circumference defined by the first top circle and one or two of the bottom circles is connected by a line tangent to those two or three circles, which line is convex. Accordingly, the entire outer shell projects the frontal orthographic plane in a purely convex fashion.

Surprisingly, the above discussed geometry will provide a breast pump having an outer shell projecting the frontal orthographic plane which provides a usable match between the demands for appropriate space for arranging components of the breast pump or the volume of e.g. the milk container within the outer shell at least in part defining the housing while meeting anatomic circumstances and demands. The present invention in particular provides a relation between different radii and distances for the centers of the first bottom circle, the second bottom circle and/or the first top circle, respectively. The absolute values may vary with the average size of the user, which average size may vary with the region looked at for specifying breast pumps to comply with the demands of a specific regional market. Nevertheless, it has turned out to be beneficial to select the radius for the first and the second bottom circle with 45 to 75 mm, preferably with 50 to 60 mm and most preferably with 55+/−2 mm. The radius of the first top circle may be selected between 30 and 46 mm, preferably may be selected within the range of 34 to 42 mm and most preferably may be 38, 1+/−2 mm.

In order to arrive at a strictly convex boundary defined by the outer shell in the frontal orthographic plane, the convex connection line which connects the outer circumference defined by at least one of the bottom circles and the top circle shall have a radius of between 58 and 94 mm, preferably between 66 and 86 mm and most preferably between 76+/−5 mm, even more preferably +/−2 mm.

As in prior art, the outer shell usually defines the outermost contour of the housing of the breast pump. This housing of the breast pump may comprise components of a hands-free motorized in-bra breast pump which motorized breast pump in its housing comprises a motor and usually a source for electric energy, like a battery, which battery may be rechargeable. The housing may furthermore comprise at least one valve which directs the fluid of expressed milk from the nipple tunnel into the milk container. The milk container of the breast pump is usually comprised within or surrounded by the outer shell. As previously mentioned with respect to prior art, the milk container may be a separate unit which is releasably attached to a housing containing the electrical components and a control for controlling the motor or a pump. The milk container may at least in part form part of the outer shell. The milk container may as well be formed by joining and sealing the outer shell and the breast shield element. In such a breast pump, a suction source for generating negative pressure within the breast pump may be an external suction source which is connected to the breast pump by e.g. a tube, which tube communicates the suction pressure of the breast pump to an internal volume surrounded by the housing, most specifically the outer shell.

The present invention preferably also defines the contour of the outer shell which projects a side orthographic plane, which side orthographic plane is perpendicular to the frontal orthographic plane. In general, the contour projecting the side orthographic plane corresponds to the contour when viewing the breast pump in a side view, whereas the projection of the outer shell of the frontal orthographic plane corresponds with the contour of a front view. In such front view, the breast pump rests with its rear side on a plane surface. When using the breast pump, the rear side is directed to the body of the user, whereas the front side is opposite thereto. When using the breast pump, the longitudinal symmetrical line may not be arranged parallel to the sagittal plane. Two breast pumps being used for expressing milk simultaneously from the left and the right breast usually intersect with their longitudinal symmetrical line at or below the level of the belly. In other words, the two breast pumps are usually slightly inclined, such that the first top circles of the two pumps are spaced apart further than the middle point or the point associated with the two centers of the bottom circles.

In a side orthographic plane, the outer shell is defined preferably by a boundary comprising a generally convex section and a generally concave section intersecting at two points. The boundary at the generally convex section may be an idealized, strictly convex approximation to the contour of the outer shell. The generally concave section defines the rear side of the outer shell, whereas the generally convex section defines the front surface projecting from the rear surface in the side orthographic plane. The contour or curvature of the generally concave section may predominantly or exclusively be defined by a flange of a breast shield, which flange rest against the breast of a user.

The generally convex section predominantly follows a curvature defined by a logarithmic spiral. Predominantly means that at least 50%, preferably 60% and most preferably 70% of this generally convex section follows the curvature defined by the logarithmic spiral. The remaining portion of the generally convex section not defined by the logarithmic spiral may have a convex constitution, a concave constitution and/or a straight, i.e. linear, constitution. Specifically, the linear section shall assume a portion of the generally convex section of less than 25%, more preferably of less than 50%. In other words, if the length of the convex section, i.e. the length between the two points in which the concave section intersects with the convex section, is 100%, the linear section preferably is not more than 50% of this total length most preferably is not more than 30% of this total length.

The linear section may e.g. be provided essentially at the apex of the outer shell. In other words, the linear section will flatten the outer shell projecting the side orthographic plane at the outermost distance from the generally concave section. The linear section may be provided above the middle point or a point associated with the centers of both first and second bottom circles. In other words, a line normal to the plane defined by the longitudinal symmetry line and the lateral symmetry line and intersecting the point associated with the two centers or the middle point will intersect with the linear section. Naturally and when viewing the breast pump in a frontal orthographic view, the outer shell defines a flat surface, which flat surface may extend essentially in the same plane as the plane defined by the lateral symmetry line and the longitudinal symmetry line. Such flat front surface may be used to support the breast pump on a table or the like in a stable position, such that e.g. a spout or vent opening of the milk container assumes the highest point.

In a respective fashion, a flat surface and thus a linear section of the outer shell projecting the side orthographic plane may be defined at the bottom of the breast pump to provide such flat support surface. In such an event, the flat bottom surface may extend essentially perpendicular to the plane defined by the lateral symmetry line and the longitudinal symmetry line.

Preferably, the linear section is encircled by the logarithmic spiral. Thus, the straight, i.e. linear contour of the outer shell projecting the side orthographic plane lies within a purely convex contour containing the logarithmic spiral and encircling the at least one linear section and possibly at least one concave section. A concave section may be formed by the outer shell and thus may form part of a housing surrounding the components of the breast pump and/or the milk container. A concave section may be adapted to receive a plug or the like, which plug when being mounted with its outer surface lies within or near the convex contour predominantly defining the outer shell projecting the side orthographic plane. Generally, any concave contour within the generally convex section usually is provided to receive a functional element like e.g. a plug or another connector, which functional element when being placed within the concave section will form part of the convex constitution of the generally convex section.

According to a preferred embodiment, a convex section length of the general convex section is between 1.5 and 2 times, preferably 1.6 and 1.8 times a concave section length of the generally concave section. The length is provided by the contour of the outer shell projecting the side orthographic plane. Respective length will follow the convex portion of the boundary irrespective of whether the contour of the shell is provided by convex, concave, or straight sections of the respective generally concave section and/or the generally convex section. The convex extension of the generally convex section is considerably larger than the concave extension of the generally concave section.

For the further definition of the outer shell projecting the side orthographic plane, a connection line is defined, which connection line is straight and connects the two intersecting points. A maximum point of the generally convex section at a direction normal to this connection line is arranged distant of the connection line by a length of between 0.4 and 0.6 of the connection line length. This maximum point may be defined by an apex within the convex geometry projecting the side orthographic plane. This maximum point may likewise be defined by the flat front surface. In the latter case, the distance is usually between 0.4 and 0.45 the connection line length from the line.

### BRIEF DESCRIPTION OF THE DRAWINGS

Further details and advantages of the breast pump of the present invention may be apparent from the following description of embodiments along with the Figures. In the drawings:

FIG. 1 shows the contour of the outer shell of a first embodiment projecting a frontal orthographic plane;

FIG. 2 shows the contour of the outer shell of a second embodiment projecting a frontal orthographic plane;

FIG. 3 is the contour of an outer shell of a first embodiment projecting the side orthographic plane;

FIG. 4 is a further drawing elucidating the embodiment of FIG. 3;

FIG. 5 correlates the contour of the outer shell projecting the side orthographic plane with the contour of a golden spiral;

FIG. 6 is the contour of an outer shell of a second embodiment projecting the side orthographic plane;

FIG. 7 is a further drawing elucidating the embodiment of FIG. 6;

FIG. 8 correlates the contour of the outer shell projecting the side orthographic plane with the contour of a golden spiral;

FIG. 9 is a view in accordance with FIG. 2 for explaining relations of some dimensions of the contour of the outer shell projecting the frontal orthographic plane;

FIG. 10 is a cross-sectional view essentially for an embodiment according to FIGS. 7 through 8; and

FIG. 11 is a side view of a breast pump containing the outer shell of the second embodiment.

FIG. 10 shows a cross-sectional view of a breast pump 2 comprising an outer shell 4 and a breast shield element 6 comprising a nipple tunnel 8, which nipple tunnel 8 is received within the outer shell 4.

### DETAILED DESCRIPTION OF THE PREFERRED EMBODIMENTS

The outer shell 4 in fact may be formed of plural parts like an outer portion of a milk container and/or an outer portion of a housing receiving components of the breast pump like a motor and/or a pressure source and/or a rechargeable battery and/or a controller. A respective housing may have an interface for setting operational parameters of the internal pump or the motor. The outer shell 4 may as well surround a chamber adapted to receive the expressed milk and may surround a valve or a valve arrangement leading the flow of expressed milk from the nipple tunnel 8 into the chamber of the milk container.

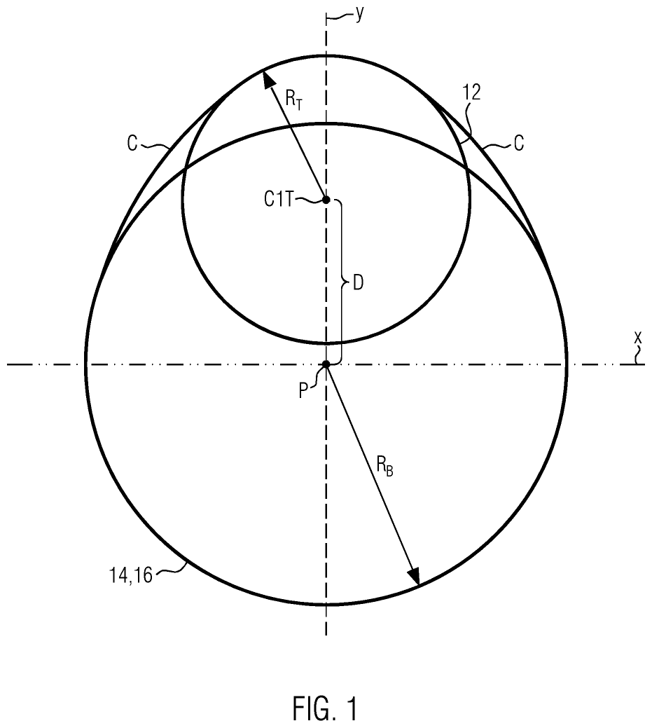

The contour of the outer shell 4 projecting a frontal orthographic plane is shown in FIGS. 1, 2, and 9. In those drawings, reference numeral 10 identifies a convex boundary, which in FIG. 1 is convex throughout the entire contour of the outer shell 4 and encircles three circles. Reference numeral 12 identifies a first top circle. Reference numeral 14 identifies a first bottom circle and reference numeral 16 identifies a second bottom circle. Those first and second bottom circles 14, 16 have the same radius R_{B}. The center of each of the circles are placed on a longitudinal symmetry line Y. A lateral symmetry line X extends perpendicular thereto. Both symmetrical lines X, Y extend within the frontal orthographic plane or are parallel thereto. Those symmetry lines X, Y have an intersecting point, which is identified with reference numeral P for the case in which the center C1B of the first bottom circle 14 is identical with the center C2B of the second bottom circle 16.

The intersection point is identified with reference numeral M in case the center C1B and the center C2B are arranged on different sides relative to the longitudinal symmetry line Y (see FIG. 2). In this FIG. 2, E identifies the distance of C1B or C2B from the longitudinal symmetry line Y. The distance E is identical for both, C1B and C2B.

D identifies the distance between the center C1T of the first top circle 12 and the inter

section point P, M.

In the embodiment of FIG. 1, the bottom portion is defined by the radius RB, whereas the top portion of the outer shell 4 is defined by the top circle 12. A convex connection line connects the radius R_{T} and the radius R_{B} in a convex fashion. The convex connection line provides a smooth transition between the two circles 14 and 16. Evidently, the contour of the outer shell 14 is symmetrical relative to the longitudinal symmetry line Y.

A respective overall contour is given for the example of FIG. 2. C1B provides the center for the bottom contour on the left-hand side of the longitudinal symmetry line Y, whereas C2B provides the center for the contour of the bottom on the right-hand side. A line extending perpendicular to the longitudinal symmetry line Y may extend as a tangent line to connect the contour provided by the first bottom circle 14 and the second bottom circle 16. Alternatively, a convex bottom connection line may be provided to render the contour of the outer shell 4 exclusively convex.

As depicted in FIG. 9, the bottom portion may be provided by a convex line having a radius R_{C} defining a bottom connection radius which bridges the slight gap at the intersection of the first and the second bottom circle 14, 16. The center of the bottom connection radius R_{C} is located on thee the longitudinal symmetry line Y. The bottom connection radius R_{C} may be 1.2 through 1.8, preferably 1.4 to 1.6, most preferably 1.5+/−10% of the first or the second bottom circle.

In FIG. 9, the first or the second bottom circle 14, 16 may be 1 or 100%. In relation thereto, the first top circle 12 is 60% or 0.6 of the bottom circle R_{B}. The distance E is 8% of the bottom circle 14 or 16. This distance D between the intersection point P/M and the center CIT of the first top circle corresponds to 70% or 0.7 of the radius R_{B}. The convex connection line C has a convex connection radius R_{CC} corresponding to 160% of R_{B}. R_{B} in the embodiment of FIG. 9 may be in the range of between 53 mm and 57 mm. R_{T} may be in the range of between 36 and 40mm. The convex connection line C can be defined by a convex connection radius of 160% of the radius of the bottom circle R_{B}.

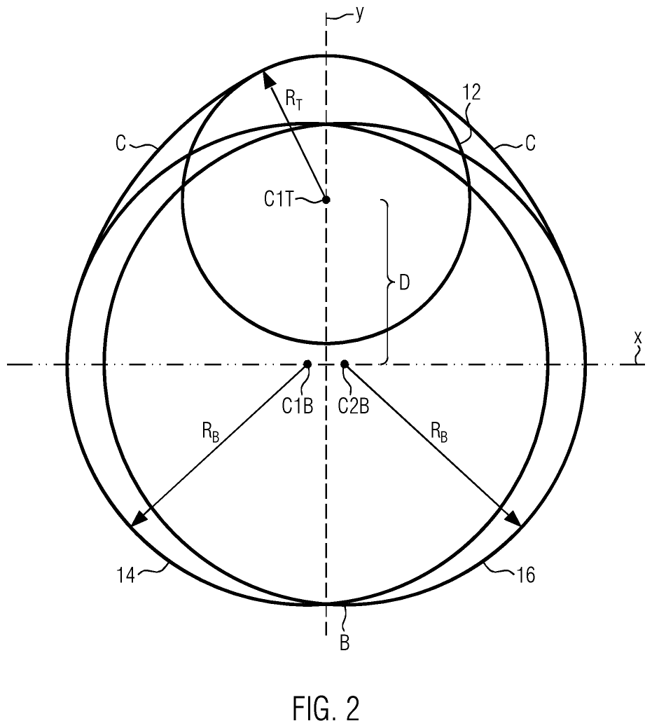

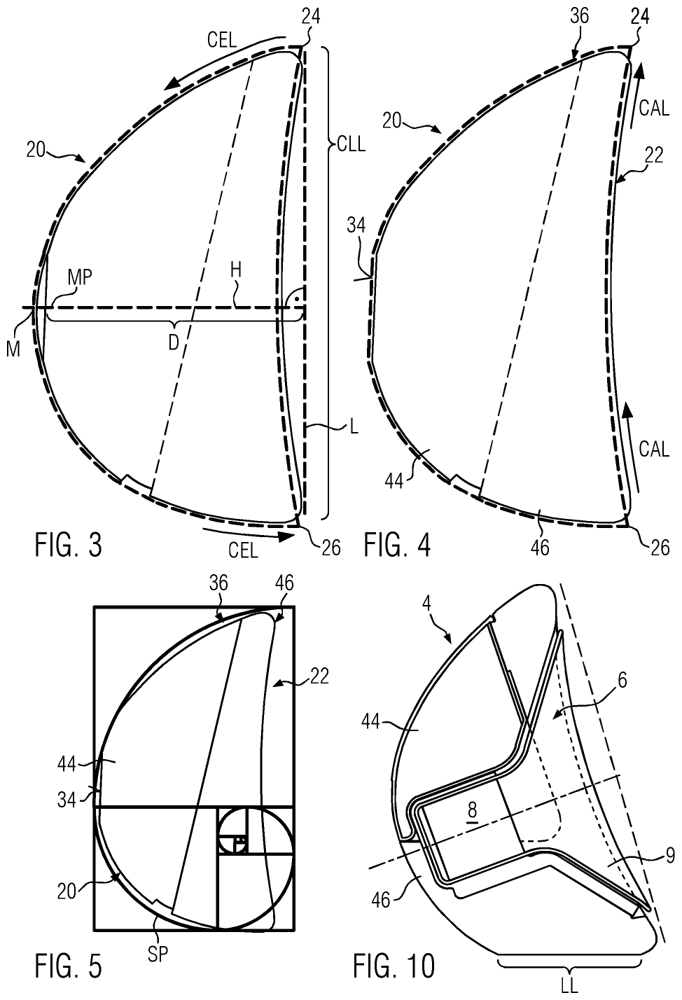

FIGS. 3 through 8 provide information on the preferred contour of the outer shell projecting a side orthographic plane. FIGS. 3 through 8 therefore show side views of examples of the present invention.

The contour generally defines a boundary with a generally convex section 20 and a generally concave section 22, which concave section 22 is provided on the rear side of the outer shell 4 and intended to be laid against the breast of the user. Those sections 20, 22 intersect at two points identified with reference numeral 24 and 26. The generally concave section 22 has a concave shape throughout its entire extension. The generally concave shape is steady and constantly but smoothly curved. In order to avoid local pressure on the body of the user exerted by the generally concave section, the same does not have any steps in the contour nor in the inclination of the concave curvature. The two points 24, 26 intersect with the contour of the generally convex section 20.

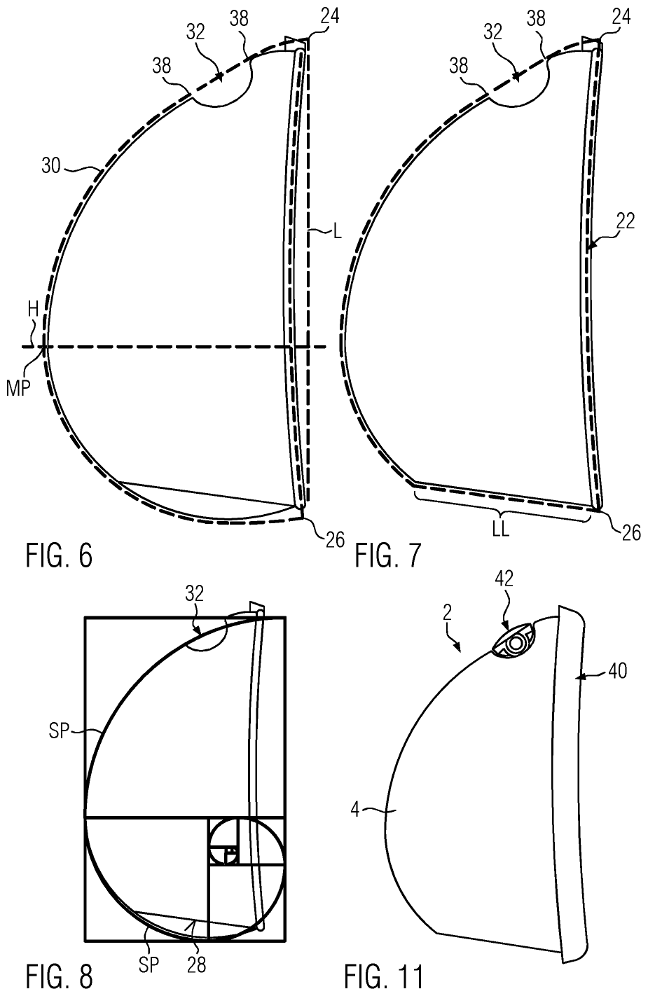

Reference numeral 24 identifies the point at the top of the outer shell 4, whereas reference numeral 26 identifies the point at the bottom of the outer shell. As evident e.g. from FIGS. 6 and 7, the lower point 26 intersects with the convex boundary 30 of the generally convex section 20. In other words, the overall convex curvature defined by the convex boundary 30 of the outer shell 4 in the side orthographic plane will have a intersecting point with the generally concave section 22. This point 26 of intersection is arranged close to a corner between the two sections 20, 22 and may be rounded. The contour of the corner is not taken into account when determining the curvature of the convex boundary 30 of the generally concave section 22. In other words, a radius below 20 mm, preferably below 10 mm of the outer shell or housing will not have an influence on the determination of the curvature of the convex boundary 30 of the generally convex section 20 and/or the generally concave section 22.

The generally concave section 22 is usually determined by the contour of a peripheral area of a flange 9 of the breast shield element 6, which flange 9 will be laid against the breast of the user and is shown e.g. in FIG. 5.

In FIGS. 6 and 7, the convex boundary 30 of the generally convex section 20 encircles a linear section 28 having a linear length LL. This linear section 28 intersects with the generally concave section 22 at point 26. Further, the convex boundary 30 intersects with the generally concave section 22 at point 26.

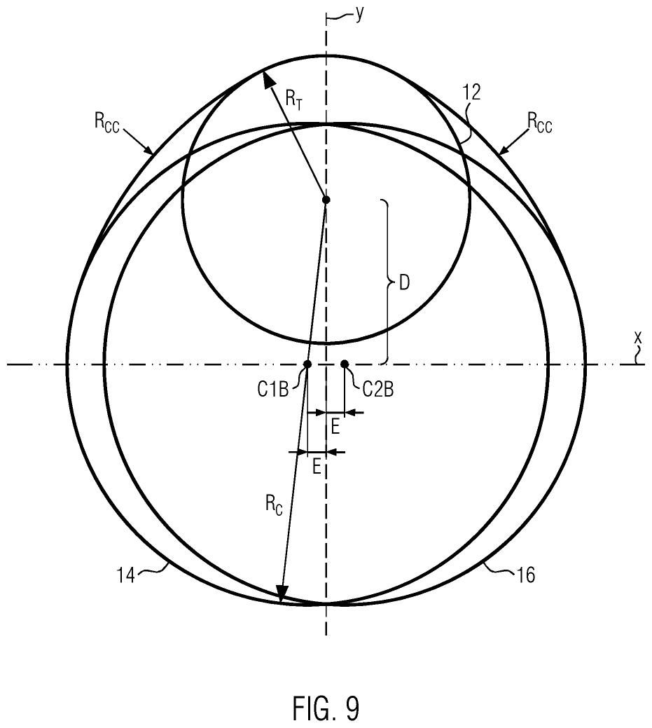

Within the overall convex boundary 30 of the outer shell 4 projecting the side orthographic plane, the outer shell 4 has a concave section 32, the borders thereof forming part of the smoothly and steadily curved convex boundary 30. The concave section 32 is to receive a plug for connecting a suction hose to the breast pump 2; See FIG. 11. In other words, in the use condition when such plug is connected, an outer surface of said plug (not shown) essentially lies within the convex boundary 30 and preferably forms part of said convex boundary 30.

This convex boundary 30 will intersect at point 24 with the generally concave section 22. As discussed before, this intersection is defined near the corner, which corner is rounded. A smaller radius will not be taken into account when determining the shape of the general convex section 20 and/or the generally concave section 22. The curvature of each of the sections 20, 22 is to be determined by an overall view of the outer shell in the side orthographic plane and/or the frontal orthographic plane in each of the cases. The convex boundary 10 or 30

Reference numeral L identifies a connection line, which is a straight line connecting the two points 24, 26. H identifies an orthogonal line. The orthogonal line H and the connection L extend parallel to the side orthographic plane. The orthogonal line H extends perpendicular to the connection line L. The orthogonal line H intersects with the generally convex section 20 at a maximum point MP. This maximum point MP may have the convex curvature of the generally convex section 20. Alternatively, and as depicted for FIGS. 3 and 4, the outer shell may provide a linear support section 34 and the intersection with the orthogonal line H, which linear support section provides a flat support surface for resting the breast pump in an upright position, e.g. on a table. With such arrangement, a spout of a milk container identified with reference numeral 36 assumes the highest point of the breast pump 2.

In FIGS. 4 and 7, reference numeral CEL identifies a convex section length. This convex section length is the distance between the two points 24, 26 along the outer, i.e. generally convex contour of the outer shell 4. CAL identifies the concave section length, which concave section length is the distance between the two points 24, 26 along the concave contour of the generally concave section 22. Evidently, the convex section length CEL also contains the length contributed to the generally convex section 20 by the linear section 28. However, any concave portion 32 is replaced by a line being tangential to each of the rim points 38 in which rim points 38, the concave section 32 intersects with the convex boundary 30.

DI identifies a distance between the intersection of the orthogonal line H and the connection line L and the intersection between the orthogonal line H and the maximum point MP.

FIGS. 5 and 8 illustrate how the outer shell 4 follows a curvature defined by a logarithmic spiral, in particular a golden spiral. “Follow” in this respect means that the convex boundary 30 of the respective outer shell 4 lies on the counter of the logarithmic spiral and/or lies within the logarithmic spiral within a range which is determined by the logarithmic spiral and lines running essentially parallel to the logarithmic spiral on both sides thereof with a distance of up to 20 mm, preferably up to 10 mm and most preferably up to 5 mm. The logarithmic spiral is identified with SP.

Evidently, the embodiment of FIG. 8 corresponds to the curvature defined by the logarithmic spiral by at least 50% of the convex boundary 30. For the embodiment of FIG. 8, in particular the bottom section, corresponds with the curvature defined by the logarithmic spiral. In the embodiment of FIG. 5, the top section up to the linear support section 34 corresponds to the curvature defined by the logarithmic spiral to a high degree. The deviation from point 24 to the linear support section 34 including this linear support section 34 is less than 4 mm for each point of the convex boundary in relation to the curvature defined by the logarithmic spiral SP.

FIGS. 6 through 8 show an outer shell which in combination with a cover will define a milk container. The respective cover is shown in FIG. 11 and identified with reference numeral 40 the inner side of the cover 40 has a curvature corresponding to the curvature of the generally concave section 22. As the cover 40 is mounted over the outer circumference of the outer shell 4, the embodiment of the breast pump exhibits a slight step near the points 22, 26. For the overall assessment of the convex/concave shape and the convex boundary, this step as well as the curved corner will not be taken into consideration. The step and the curved corner both are defined by small radii, which do not meet the radius requirement and thus will not be taken into consideration for determining the contour of the convex boundary. FIG. 11 also shows the plug element received in the convex section 32, which plug element is identified with reference numeral 42.

FIGS. 3 through 5 show the outer shell 4 which is defined by the flange 9 of the breast shield element 6, a milk container 44 and a housing 46 containing and surrounding a motor, a battery and a control. In FIG. 10, the housing 46 has an L-shaped recess, which receives the milk container 44, which milk container 44 is attached to the housing 46. The upper portion of the outer shell 4 above the hashed centerline of the nipple tunnel 8 is partially provided by the housing 46 (uppermost portion), the milk container 44 and a portion of the housing 46 (front portion) defining bottom of the L-shaped recess.

### REFERENCE SIGNS

2 breast pump
4 outer shell
6 breast shield element
8 nipple tunnel
9 flange of the breast shield element
10 convex boundary
12 first top circle
14 first bottom circle
16 second bottom circle
20 generally convex section
22 generally concave section
24 point
26 point
28 linear section
30 convex boundary
32 concave section
34 linear support section
36 spout
38 rim point
40 cover
42 plug element
B bottom connection line
C convex connection line
CEL convex section length
CAL concave section length
CLL connection line length
C1B center of the first bottom circle
C2B center of the second bottom circle
CIT center of the first top circle
D distance for MP-CIT
DI distance for MP
E distance of C1B and C2B from the intersection point P; M
H orthogonal line
L connection line
LL linear length
MP maximum point
P; M intersection point
R_{B} radius of the first or the second bottom circle
R_{C} bottom connection radius
R_{CC} convex connection radius
R_{T} radius of the first top circle
SP logarithmic spiral
X lateral symmetry line
Y longitudinal symmetry line

1. A breast pump shaped at least in part to fit inside a bra, comprising:
an outer shell and a breast shield element including a nipple tunnel adapted to receive a nipple, wherein the outer shell projects a frontal orthographic plane that is shaped like an oval, which oval is defined by a convex boundary encircling three circles, a first top circle, a first bottom circle and a second bottom circle, wherein a radius (R_{B}) of the of the first bottom circle and a radius (R_{B}) of the second bottom circle (16) are equal and the ratio of a radius (R_{T}) of the first top circle to the radius (R_{B}) of the first or the second bottom circles is between 0.3 and 0.8.

2. The breast pump according to claim 1, wherein a center (C1B) of the first bottom circle and a center (C2B) of the second bottom circle are positioned on a lateral symmetry line (X) at a distance equal to a value between 0.0% and 15.0% of the radius (R_{B}) of the first or the second bottom circle.

3. The breast pump according to claim 2, wherein a center (C1T) of the first top circle is located on a longitudinal symmetry line (Y) that orthogonally intersects the lateral symmetry line (X) at a point (P) associated with both centers (C1B; C2B) of first and second bottom circles or at a middle point (M) between the centers (C1B; C2B) of the first and second bottom circles (C1B; C2B).

4. The breast pump according to claim 3, wherein the center (C1T) of the first top circle is positioned on the longitudinal symmetry line (Y) at a distance greater than R_{B}−R_{T} and less than R_{B}+R_{T} from the intersection point (P; M) with the lateral symmetry line (X), wherein R_{T} is the radius of the first top circle and wherein R_{B} is the of one of the first or the second bottom circle.

5. The breast pump according to claim 1, wherein the convex boundary is exclusively convex.

6. The breast pump according to claim 1, wherein the outer shell projects a side orthographic plane defined by a boundary comprising a generally convex section and a generally concave section intersecting at two points.

7. The breast pump according to claim 6, wherein the generally convex section predominantly follows a curvature defined by a logarithmic spiral (SP).

8. The breast pump according to claim 7, wherein at least 60% of the generally convex section follows the curvature defined by a logarithmic spiral (SP).

9. The breast pump according to claim 7, wherein the logarithmic spiral (SP) is a golden spiral.

10. The breast pump according to claim 6, wherein the generally convex section includes a linear section, which linear section is less than 25%.

11. The breast pump according to claim 6, wherein the generally convex section has a convex section length (CEL) and the generally concave section has a concave section length (CAL), wherein the convex section length (CEL) is between 1.5 and 2.0 times the concave section length (CAL).

12. The breast pump according to claim 6, wherein the two intersecting points define a connection line (L) with a connection line length (CLL), wherein a maximum point (MP) of the generally convex section is on an orthogonal line (H) that orthogonally intersects the connection line (L) and is located at a distance between 0.4 and 0.6 of the connection line length (CLL) from the line (L).

13-16. (canceled)

17. The breast pump according to claim 8, wherein the logarithmic spiral (SP) is a golden spiral.

18. A breast pump shaped at least in part to fit inside a bra and comprising an outer shell and a breast shield element including a nipple tunnel adapted to receive a nipple, wherein the outer shell projects a side orthographic plane defined by a boundary comprising a generally convex section and a generally concave section intersecting at two points.

19. The breast pump according to claim 18, wherein the generally convex section predominantly follows a curvature defined by a logarithmic spiral (SP).

20. The breast pump according to claim 19, wherein at least 60% of the generally convex section follows the curvature defined by a logarithmic spiral (SP).

21. The breast pump according to claim 19, wherein the logarithmic spiral (SP) is a golden spiral.

22. The breast pump according to claim 18, wherein the generally convex section includes a linear section, which linear section is less than 25% of the generally convex section.

23. The breast pump according to claim 18, wherein the generally convex section has a convex section length (CEL) and the generally concave section has a concave section length (CAL), wherein the convex section length (CEL) is between 1.5 and 2.0 times the concave section length (CAL).

24. The breast pump according to claim 18, wherein the two intersecting points define a connection line (L) with a connection line length (CLL) and wherein a maximum point (MP) of the generally convex section is on an orthogonal line (H) that orthogonally interests the connection line (L) and is located at a distance between 0.4 and 0.6 the connection line length (CLL) from the line (L).
